(19) United States
(12) Patent Application Publication
(10) Pub. No.: US 2009/0111957 A1
(43) Pub. Date: Apr. 30, 2009

(54) FLUORO-PONYTAILED BIPYRIDINE DERIVATIVES AND THEIR USE AS LIGANDS IN THE METAL-CATALYZED ATRP

(75) Inventors: Norman Lu, Taipei County (TW); Tsung-Chi Chen, Taipei County (TW)

Correspondence Address: WPAT, PC, 7225 BEVERLY ST., ANNANDALE, VA, 22003, US

(73) Assignee: NATIONAL TAIPEI UNIVERSITY OF TECHNOLOGY, Taipei City (TW)

(21) Appl. No.: 11/930,402

(22) Filed: Oct. 31, 2007

(51) Int. Cl. C08F 4/06; C07D 401/04; C07F 15/00

(52) U.S. Cl. 526/172; 546/10; 546/258

(57) ABSTRACT

The present invention also relates a metal complex complexing with the fluoro-ponytailed bipyridine derivatives, which is represented by the general formula (2):

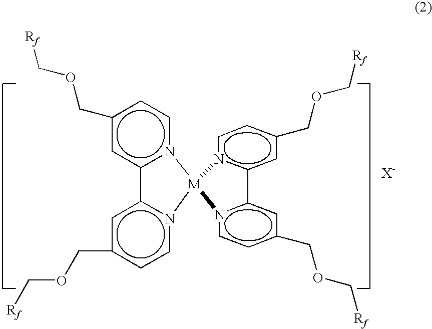

and each R_{f} is the same or different and represents a fluoro-alkyl group having from 3 to 11 carbon atoms, preferably a perfluoro-alkyl group having from 9 to 11 carbon atoms; X⁻ represents a halogenide such as fluoride, bromide, chloride, or iodide; and M represents a metal selected from the group consisting of Mo, Cr, Re, Ru, Fe, Rh, Ni, Pd, and Cu.

### FIELD OF THE INVENTION

The present invention relates to fluoro-ponytailed bipyridine derivatives and their use as ligands in the metal-catalyzed atom transfer radical polymerization (ATRP).

### BACKGROUND OF THE INVENTION

The search for recoverable catalysts is a major concern in the field of catalysis (Gladysz, J. A., Guest Ed. Chem. Rev. 2002, 102, 3215). Atom transfer radical polymerization (ATRP) is an area of intense research because of the possibility of controlling the molecular weight, poly-dispersity index (PDI) and the end-functionalized synthesis of the final polymer (Tsarevsky, N. V.; Matyjaszewski, K. Chem. Rev. 2007, 107, 2270). Unfortunately, ATRP typically uses one metal/ligand complex to mediate one growing polymer chain to achieve reasonable reaction rates. Consequently, the resulting polymer is colored because of the residual metal.

Indeed, one of the limitations of ATRP for its industrial development is the presence of residual transition metal catalyst in the final polymer which may cause environmental problems. Different purification methods were proposed in the recent literature, among which the most developed is the immobilization of the ATRP catalyst onto organic or inorganic polymeric supports (J. V. Nguyen, C. W. Jones, Journal of Catalysis 2005, 232 (2), 276). However, the immobilized catalysts often do not effectively mediate the polymerization process. This may be attributed to a number of possible reasons, including poor access of the growing radical chain end to deactivating species (Queffelec, J.; Gaynor, S. G.; Matyjaszewski, K. Macromolecules 2000, 33, 8629) or catalyst heterogeneity (Haddleton, D. M.; Kukulj, D.; Radigue, A. P. Chem. Commun. 1999, 99; Kickelbick, G.; Paik, H.-J.; Matyjaszewski, K. Macromolecules 1999, 32, 2941; Haddleton, D. M.; Duncalf, D. J.; Kukulj, D.; Radigue, A. P. Macromolecules 1999, 32, 4769).

Recently, more efficient purely heterogeneous catalysts (Nguyen, J. V.; Jones, C. W. Macromolecules 2004, 37, 1190; Shen, Y.; Zhu, S.; Zeng, F.; Pelton, R. H. Macromolecules 2000, 33, 5427; Shen, Y.; Zhu, S.; Pelton, R. Macromolecules 2001, 34, 5812), two component heterogeneous/homogeneous catalysts (Hong, S. C.; Paik, H.-J.; Matyjaszewski, K. Macromolecules 2001, 34, 5099; Hong, S. C.; Matyjaszewski, K. Macromolecules 2002, 35, 7592; Yang, J.; Ding, S.; Radosz, M.; Shen, Y. Macromolecules 2004, 37, 1728.), or thermoresponsive catalysts (Shen, Y.; Zhu, S.; Pelton, R. Macromolecules 2001, 34, 3182) were reported. However, the relatively tedious preparation and recovery procedures might pose limitations for the industrial applications. In 1999, Vincent et al. (De Campo, F.; Lastecoueres, D.; Vincent, J.-M.; Verlhac, J.-B. J. Org. Chem. 1999, 64, 4969) reported the first example of a molecular recyclable catalyst for ATRP that was based on the thermomorphic behavior of a fluorous biphasic system (FBS), which was proved to be effective for catalyst recovery in ATRP. However, its expensive cost and its low efficiency in controlling the molar masses of the polymers prevent it from the industrial applications (Haddleton, D. M.; Jakson, S. G.; Bon, S. A. F. J. Am. Chem. Soc. 2000, 122, 1542).

Gladysz and co-workers recently introduced the solubility-based thermomorphic properties of heavy fluorous catalysts in organic solvents as a new strategy to perform the homogeneous catalysis without fluorous solvent (Wende, M.; Meier, R.; Gladysz, J. A. J. Am. Chem. Soc. 2001, 123, 11490; Wende, M.; Gladysz, J. A. J. Am. Chem. Soc. 2003, 125, 5861). Catalyst recovery was achieved by an easy liquid/solid separation (Shen, Z.; Y. Chen, Y.; H. Frey, H.; Stiriba, S.-E. Macromolecules 2006, 39, 2092). Vincent et al. in 2004 also reported the solubility-based thermomorphic properties of non-fluorous catalyst which is based on the long hydrocarbon chain (C₈H₁₇) (G. Barre, D. Taton, D. Lastecoueres, J.-M. Vincent, J. Am. Chem. Soc. 2004, 126, 7764). Inspired by these works, the present inventors wondered whether or not the approach could be extended, for particular cases, to catalysts in which the perfluoroalkylated bipyridine chains were used. Therefore, the present inventors have investigated the thermormorphic advantages of homogeneous catalysis at an elevated temperature and simple recovery by solid/liquid decantation at room temperature and thus completed the present invention.

### SUMMARY OF THE INVENTION

The present invention relates to a fluoro-ponytailed bipyridine derivatives represented by the general formula (1):

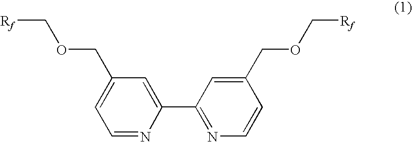

wherein:
each R_{f} is the same or different and represents a fluoro-alkyl group having from 3 to 11 carbon atoms, preferably a perfluoro-alkyl group having from 9 to 11 carbon atoms.

The fluoro-ponytailed bipyridine derivatives (1) of the present invention are useful as ligands of a metal complex such as copper complex. After forming a metal complex with a metal, the fluoro-ponytailed bipyridine derivatives of the present invention exhibit a property of dissolving in solvents at an elevated temperature but solidifying in the solvents at room temperature, so that the metal complex containing the fluoro-ponytailed bipyridine derivatives (1), when being used a catalyst in atom transfer radical polymerization (ATRP), is easily separated and recovered effectively from the resultant polymer by simply solid/liquid decantation at room temperature. Therefore, no or few residual catalyst remains in the final polymer.

The present invention also relates a metal complex complexing with the fluoro-ponytailed bipyridine derivatives, which is represented by the general formula (2):

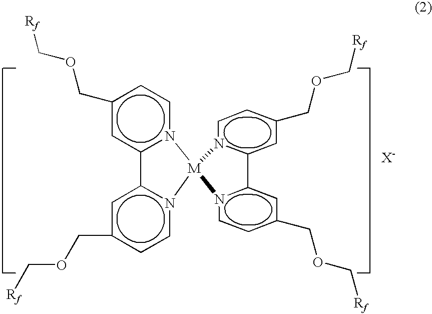

wherein:
each R_{f} is the same or different and represents a fluoro-alkyl group having from 3 to 11 carbon atoms, preferably a perfluoro-alkyl group having from 9 to 11 carbon atoms;
   X⁻ represents a halogenide such as fluoride, bromide, chloride, or iodide; and
   M represents a metal selected from the group consisting of Mo, Cr, Re, Ru, Fe, Rh, Ni, Pd, and Cu.

The present invention also relates to a method for polymerizing vinyl-containing monomers, which comprises the steps of: (a) polymerizing one or more of vinyl-containing monomers by using the metal complex (2) having the fluoro-ponytailed bipyridine derivatives (1) as a catalyst at elevated temperature, and (b) separating the metal complex (2) from the reaction mixture by cooling the temperature of the mixture down to room temperature.

In the present method, the polymerization of one or more of vinyl-containing monomers is an atom transfer radical polymerization (ATRP) under the thermomorphic mode.

In the present method, the vinyl-containing monomer is selected from the group consisting of alkyl acrylate, alkyl methacrylate, styrenes, and derivatives thereof.

In the present method, the polymerization is carried out at a temperature of from 40˜120° C.

In the present method, the polymerization is carried out in the presence of initiator. Examples of the initiator include those conventional used in atom transfer radical polymerization, for example, but are not limited to, ethyl 2-bromoisobutyrate, (1-bromoethyl)benzene, 1-bromoacetonitrile, 2-bromopropionitrile, Azobisisobutyronitrile (AIBN), and the like.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1 shows the controlled result in the system CuBr/1b (wherein R_{f} represents n-C₁₀F₂₁) catalyzed ATRP (atom transfer radical polymerization) of MMA in two different concentrations at 80° C.

FIG. 2 shows a kinetic plot of CuBr/1a-c complexes catalyzed ATRP wherein ▪□ represents CuBr/1a, ◯ represents CuBr/1b, and ▴□ represents CuBr/1c; ▪ [symbol] ▴: a plot of time vs. the conversion; □⋆Δ: a plot of time vs. ln(Mₒ/M)].

FIG. 3 shows the plot of the molecular weight and PDI vs. conversion for systems wherein ▪□ represents CuBr/1a, ◯ represents CuBr/1b, and ▴□ represents CuBr/1c; [▪ [symbol] ▴: a plot of conversion vs. the molecular weight; □⋆Δ: a plot of conversion vs. PDI (polydispersity index)].

FIG. 4 shows a plot of conversion vs. the molecular weight (or PDI) by CuBr/1a system for the ATRP of MMA in which ▪□: slow addition of initiator in 5 min; [symbol] ⋆: halogen exchange by adding CuCl; and ▴Δ: adding the 10% deactivating agent, CuBr₂; [▪ [symbol] ▴: a plot of conversion vs. the molecular weight; □⋆Δ: a plot of conversion vs. PDI (polydispersity index)].

FIGS. 5(a) and 5(b) are photographs showing that the precipitated Cu complex (2) catalyst being easily separated from the product mixture.

FIG. 6 is photograph showing that the colorless PMMA obtained with evaporation of solvent after decantation.

FIG. 7 is a photograph showing that the recovery of metal complex (2) after ATRP reaction.

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

In the fluoro-ponytailed bipyridine derivatives of the present invention, the fluoro-alkyl group having from 3 to 11 carbon atoms represented by R_{f} means a straight- or branched alkyl having 3 to 11 carbon atoms in which one or more hydrogen atoms are replaced with fluoro atom(s), preferably all hydrogen atoms are replaced with fluoro atoms. More preferably, the fluoro-alkyl group is that having from 9 to 11 carbon atoms in which one or more hydrogen atoms are replaced with fluoro atom(s), preferably all hydrogen atoms are replaced with fluoro atoms. The metal complexes of the present invention are insoluble in solvents at room temperature but soluble in the solvent when temperature is moderately raised so that it can form homogeneous phase in reaction mixture. After the end of reaction, the metal complex can be easily separated from the reaction mixtures by cooling the temperature down since the metal complexes will precipitate again. Thus, we can easily separate the metal complexes from polymers by simple liquid/solid method.

In the present invention, the vinyl-containing monomer to be polymerized through the use of the present metal complex (2) having the fluoro-ponytailed bipyridine derivatives (1) as catalyst can be any monomer as long as it possesses one or more vinyl group and is (co)-polymerized through the atom transfer radical polymerization (ATRP). Examples of the vinyl-containing monomer include, but are not limited to, alkyl acrylate, alkyl methacrylate, unsubstituted or substituted styrenes, and derivatives thereof; for example, methyl acrylate, ethyl acrylate, n-propyl acrylate, butyl acrylate, pentyl acrylate, hexyl acrylate, methyl methacrylate, ethyl methacrylate, n-propyl methacrylate, butyl methacrylate, pentyl methacrylate, hexyl methacrylate, styrene, α-methyl styrene, and the like.

The preparation of the fluoro-ponytailed bipyridine derivatives of the present invention is illustrated by the following scheme:

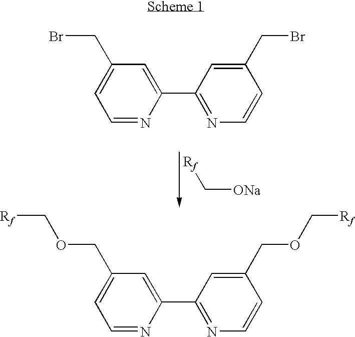

wherein:
each R_{f} is the same or different and represents a fluoro-alkyl group having from 3 to 11 carbon atoms, preferably a perfluoro-alkyl group having from 9 to 11 carbon atoms.

As shown in Scheme 1, the preparation of the fluoro-ponytailed bipyridine derivatives started from deprotonation of readily available fluorous alkanols, R_{f}CH₂OH, wherein R_{f} is defined as above. Fluorous alkanols, R_{f}CH₂OH, were treated with CH₃ONa solution (30% in CH₃OH) to give the corresponding alkoxides (3). The alkoxides (3) were then reacted with 4,4′-bis(BrCH₂)-2,2′-bipyridine (1) (prepared as mentioned in Ciana, L. D.; Dressick, W. J. J. Heterocyclic Chem. 1990, 27, 163; Oki, A. R.; Morgan, R. J. Synth. Commun. 1995, 25, 4093; and Will, G.; Boschloo, G.; Rao, S, N.; Fitzmaurice, D. J. Phys. Chem. B, 1999, 103, 8067) to give the fluoro-ponytailed bipyridine derivatives (1).

The metal complex (2) can be generated in situ by stirring the fluoro-ponytailed bipyridine derivatives (1) with metal halogenide such as bromides, chlorides of Mo, Cr, Re, Ru, Fe, Rh, Ni, Pd, and Cu, for example CuBr, in a mole ratio of from 2:1 to 6:1 under inert gas, preferably under nitrogen atmosphere. The solubility of metal complex (2), especially Cu complex (2), in toluene increases about 500-fold when the temperature was raised from 20° C. to 80° C. Interestingly, ligands—the fluoro-ponytailed bipyridine derivatives (1)—were found to be useful for the ATRP of vinyl-containing monomer in solvent under the thermomorphic mode. The temperature dependent solubility of metal complexes (2), for example Cu complex (2), was determined by the recrystallization method; both CuBr (0.05 mmol, 7.15 mg) and ligand 1a (wherein R_{f} represents n-C₉F₁₉) (0.1 mmol, 118 mg) were combined to first make the CuBr/ligand 1a (hereinafter sometimes refer to CuBr/1a) system which was dissolved in toluene (added with little bit of DMF to fasten the process) to make 10 mM and 0.02 mM solutions. These two solutions (10 mM and 0.02 mM) were not soluble in toluene at 20° C. However, both solutions were soluble in toluene at 80 (±3)° C. Therefore, it is known that the solubility of the CuBr/1a system increased 500-fold (10/0.02=500) when the temperature was raised from 20 to 80° C.

Take ligand 1b (wherein R_{f} represents n-C₁₀F₂₁) for example, the system CuBr/ligand 1b (hereinafter sometimes refer to CuBr/1b) was prepared from CuBr and ligand 1b at a mole ratio of 1:2 (CuBr: ligand 1b). And the mixture was stirred in the co-solvent (acetonitrile/FC-77 (a distilled mixture of perfluorinated solvent whose boiling point range is close to n-C₈F₁₈ and is commercially available from 3M Company, U.S.A.)/HFE-7100 (perfluorobutyl methyl ether; C₄F₉OCH₃)) for 8 h under nitrogen atmosphere. The CuBr/1 complex (also refer to Cu complexes 2) was easily isolated as a dark color solid under the nitrogen atmosphere because the CuBr/1 complexes are known to be sensitive to molecular oxygen. The ATRP of methyl methacrylate (MMA) was carried out in toluene at 80° C. using ethyl 2-bromoisobutyrate as an initiator and CuBr/1 [1a (wherein R_{f} represents n-C₉F₁₉), 1b (wherein R_{f} represents n-C₁₀F₂₁) or 1c (wherein R_{f} represents n-C₁₁F₂₃)] as the catalyst. The preparation of the ligands 1a, 1b, 1c, and the Cu complexes are shown in examples hereinafter.

The ATRP mechanism, shown in Scheme 2, included the equilibrium of Cu complexes and the polymerization/termination reactions. The order of K values of the 3 equilibria should be K₁<K₂<K₃ because once the complexes CuBr/1a-1c form at right, the most bulky species CuBr/1c is the most difficult one to undergo the backward reaction to return to the complex CuBr/1c sterically.

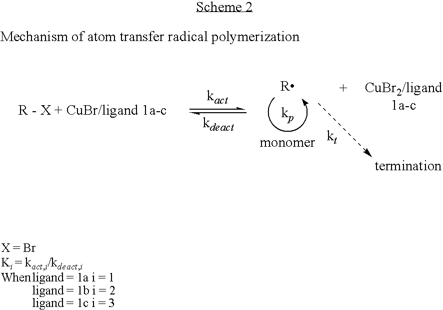

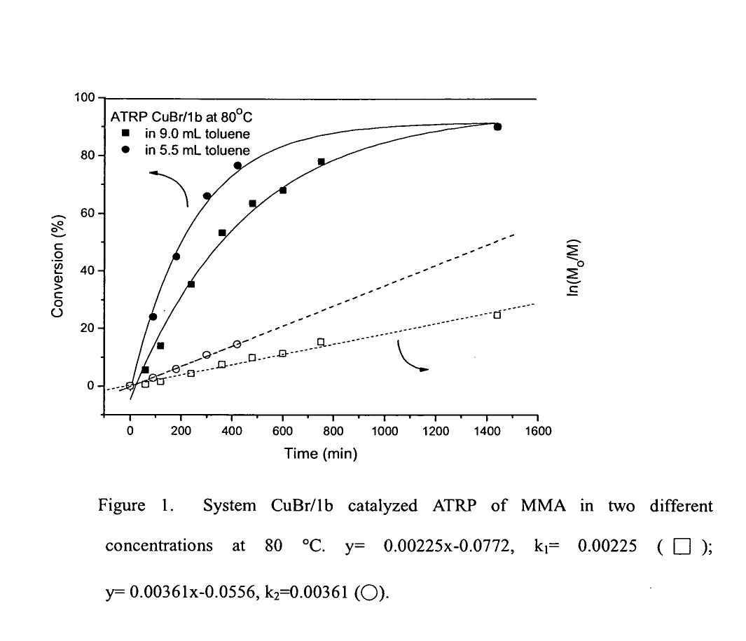

When the system CuBr/1b was used for the atom transfer radical polymerization (ATRP) in toluene at the different concentrations, the controlled results were obtained as shown in FIG. 1. The rate of the same amount of monomer (1 g; ca. 1 mL) catalyzed by CuBr/1b system in 9 mL toluene was ca 0.65 [=1+9/(1+5.5)] times slower than that in 5.5 mL toluene. The ratio of rate constants, k₁ and k₂, from two different concentrations was also close to 0.65 as shown in FIG. 1.

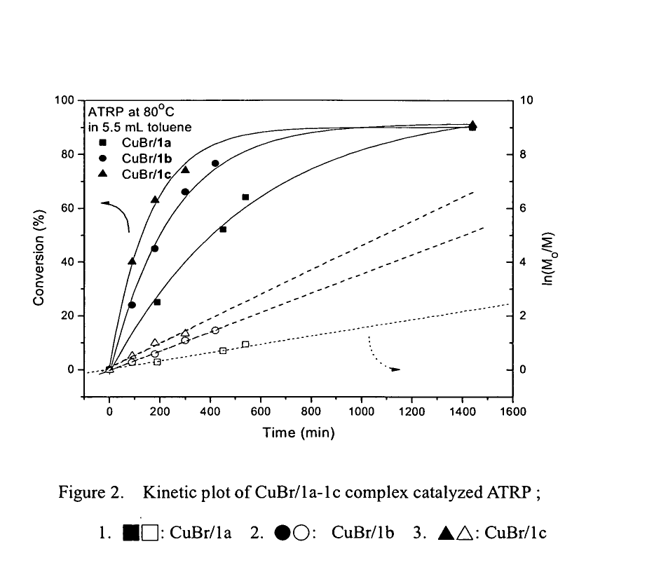

At 80° C. the preformed molecular CuBr/1a-1c complexes (also refer to Cu complexes 2a-2c) were soluble, allowing precise control of the amount of catalyst present in solution at the early stage of the reaction to ensure an efficient initiation step. Furthermore, the all three polymerizations whose conversions were all close to 90% within 24 h proceeded efficiently at 80° C. with first-order kinetics with respect to monomer concentration (FIG. 2). The reaction rates as shown were system CuBr/1c>system CuBr/1b>system CuBr/1a because system CuBr/1c with the longest fluorinated chain could make k_{act}/k_{deact} value, due to the steric reason, largest among the three and the concentration of radical was then increased. And the ln (M₀/M) was linearly dependent on time.

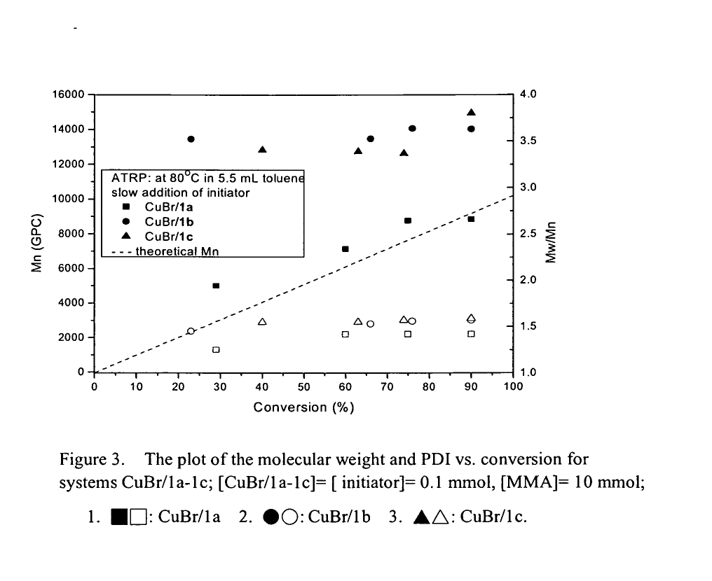

FIG. 3 shows the plot of the molecular weight and PDI vs. conversion for systems. As shown in FIG. 3, the number averaged molecular weight (Mₙ) and the polydispersity index (PDI) results of resulting PMMA from CuBr/1a-1c systems were plotted against conversion; the initiator being added within 5 min during the polymerizations. The CuBr/1a catalyzed ATRP of MMA had the lowest PDI, the reasonably controlled molecular weight (MW) and initiation efficiency. The CuBr/1a catalyzed reaction was the slowest among the three systems, taking ca. 24 h to reach the 90% conversion level. The relatively high concentration of radicals (R.) in the CuBr/1b or CuBr/1c catalyzed ATRP made the control of MW and MW distribution not as good as those obtained in the CuBr/1a catalyzed ATRP.

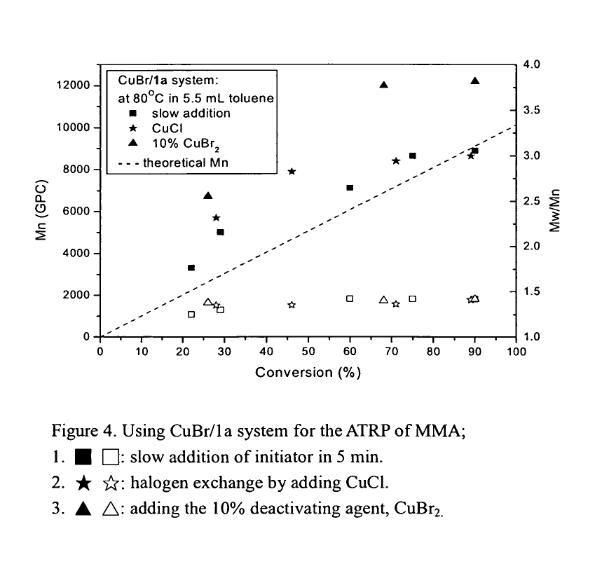

In addition to the theoretical number averaged molecular weights, the plots of molecular weight versus conversion for the CuBr/1a catalyzed ATRP with 3 different methods were shown in FIG. 4. In the 1st method, the initiator was slowly added into the reaction mixture within 5 min to ensure the generation of enough radicals at the beginning of the initiation. The plot of Mₙ vs. conversion from this method was linear and close to the theoretical prediction. The slow addition data showed a good control of the molar masses of the polymers, with fairly narrow PDI of the resulting PMMA, in the range of 1.26 and 1.41. The initiation efficiency of system CuBr/1a was also very close to 100%. Furthermore, the 2nd method was to use the halogen exchange technique, adding CuCl instead of CuBr to mediate the reaction (Matyjaszewski, K.; Wang, J. L.; Grimaud, T.; Shipp D. A. Macromolecules 1998, 31, 1527-1534; Matyjaszewski, K.; Shipp, D. A.; Wang, J. L.; Grimaud, T.; Patten, T. E. Macromolecules 1998, 31, 6836-6840). Lastly, the 3rd method was to add the 10% deactivating agent, CuBr₂, to control the polymerization (Zhang, H.; Klumperman, B.; Ming, W.; Fischer, H.; van der Linde, R. Macromolecules, 2001, 34, 6169-6173). The results of the 2^{nd} or 3rd method were not as good as those of the 1st method for CuBr/1a catalyzed ATRP of MMA.

During the work-up, the product solution was cooled down to −10° C. in the freezer, then followed by centrifugation, and the precipitated Cu complex catalyst being easily separated from the product mixture (FIG. 5). The used CuBr/1a-1c complexes were then simply recovered by centrifugation (>99% yield). After evaporation of the volatiles, PMMA was obtained from the colorless filtrate as a white glassy solid without further purification (FIG. 6). Furthermore, a block copolymer consisting of MMA units as the first block and butyl methacrylate (BMA) as the second block was successfully prepared by chain-extending a PMMA precursor. When PMMA-macro-initiator (Mn: 8900, PDI=1.41) and BMA were used for the copolymerization, a block copolymer of p(MMA-b-BMA) was also successfully isolated. The preliminary results showed that the yield of copolymer p(MMA-b-BMA) analyzed by ¹H NMR was 73% and its MW was 21500. This result successfully demonstrated the living character of the CuBr/1a catalytic system.

TABLE 1
The amount of residual Cu determined by ICP-MS

| | Amount of residual Cu | |
|---|---|---|
| Cu catalyst | (ppm)^{a} | Recovery (%) |
| CuBr/1a | 19.3 | 99.73 |
| CuBr/1b | 14.3 | 99.80 |
| CuBr/1c | 39.4 | 99.45 |

^{a}the detection limit of ICP-MS is 0.07 ppm.

Inductive coupled plasma (ICP) analysis revealed the low amounts of residual copper in the polymers when catalyzed by three CuBr/1a-1c systems. These results were summarized in Table 1. Because the ATRP of MMA catalyzed by CuBr/1a system demonstrated the best control in terms of PDI, the conversion and MW relationship and initiation efficiency, we used the data obtained by the CuBr/1a system catalyzed ATRP as an example and did some calculations and comparisons. The 19.3 ppm was the amount of residual Cu detected by the ICP-MS when the polymerization was catalyzed by CuBr/1a. This 19.3 ppm which could be even lower if the resulting PMMA was formed by adding the excess methanol to cause precipitation, showed a low Cu content as opposed to 7044 ppm expected if all the catalyst remained in the polymer. As indicated in Table 1, the amount of recovered Cu was as high as 99.73% for recycling CuBr/1a catalyst. And 19.3 ppm was much lower than 200 ppm reported for the non-fluorous thermoresponsive system (G. Barre, D. Taton, D. Lastecoueres, J.-M. Vincent, J. Am. Chem. Soc. 2004, 126, 7764). The recovered catalyst was difficult to be reduced and reused. However, the preliminary results showed that the used catalyst could be used for the reverse ATRP of MMA. [supporting information; reverse ATRP as below]. Furthermore, the more expensive ligand 1a-1c could be recycled with 74-84% yield by adding the excess aqueous EDTA (ethylene diamine tetra-acetate) solution to the used Cu complex (2) which was dissolved in fluorinated solvent (e.g. FC 77) and stirring at room temperature for several days [supporting info; FIG. 7].

To conclude, a series of novel fluorinated bipyridine ligands (1a-1c) were prepared with good yields. The easiness of preparation and handling, the good conversion of polymerization and the recovery of complexes by simple filtration in air, the reverse ATRP by the used Cu complexes, and the very low contents (less than 0.6%) of residual metal in the final polymers make the CuBr/1a-1c catalysts (Cu complexes (2)) with the novel fluorinated ligands 1a-1c the effective systems for living radical polymerization of MMA under the thermomorphic mode. Additionally, these results show that for catalytic reactions performed in toluene, introduction of fluoro-ponytailed bipyridine catalysts might be considered as a valuable strategy to achieve the recovery by simple liquid/solid decantation and obtain the well-controlled living polymers. In particular, the ATRP catalyzed by CuBr/1a system showed the well-controlled polymerization, narrow PDI and low residual metal content. These properties could make the ATRP one step closer to the industrial applications.

The present invention is now described in more detail by reference to the following examples. The examples are only used for illustrating the present invention without limiting the scope of the present invention.

### EXAMPLES

### Example 1

### Preparation of 4,4′-bis(R_{f}CH₂OCH₂)-2,2′-bipyridine (1a)-(1c) wherein R_{f}=n-C₉F₁₉ (1a), n-C₁₀F₂₁ (1b), n-C₁₁F₂₃ (1c)

General procedure: 30% CH₃ONa/CH₃OH (15.0 mmol) and R_{f}CH₂OH (15.0 mmol) were charged into a round-bottomed flask, then continuously stirred under N₂ atmosphere at 65° C. for 4 h before CH₃OH was vacuum removed to drive the reaction to the fluorinated alkoxide (R_{f}CH₂ONa) side. The resultant fluorinated alkoxide (15.0 mmol) was then dissolved in 20 mL of dry THF, and 4,4′-bis(BrCH₂)-2,2′-bipyridine (5.8 mmol, 2 g) was added. The mixture was brought to reflux for 4 h, and the completeness of the reaction was checked by sampling the reaction mixtures and analyzing the aliquots with GC/MS. The product was purified by vacuum sublimation to obtain white solids. The vacuum level was 0.1 torr, and the sublimation temperature was 50° C. above its m.p.

Compound 1a: yield (sublimed) 72%; ¹H NMR (500 MHz, D-toluene) δ 8.51 (2H, d, ³J_{HH}=4.7 Hz, H₆), 8.53 (2H, s, H₃), 6.93 (2H, d, ³J_{HH}=4.7 Hz, H₅), 4.18 (4H, s, bpy-CH₂), 3.56 (4H, t, ³J_{HF}=13.5 Hz, CF₂CH₂); ¹⁹F NMR (470.5 MHz, D-toluene) δ −80.8 (3F), −118.7 (2F), −121.8 (8F), −122.6 (2F), −123.2 (2F), −125.6 (2F); ¹³C NMR (113 MHz, D-toluene) δ 73.5 (bpy-CH₂), 68.2 (CH₂CF₂), 119.7, 121.9, 146.9, 149.9, 157.2 (bpy), 105.0˜116.0 (C₈F₁₇); GC/MS (m/z; EI): 682 (M⁺-OCH₂C₉F₁₉), 198 (C₅H₃NCH₂C₅H₃NCH₂O⁺), 183 (C₅H₃NCH₂C₅H₃NCH₃⁺), 91 (C₅H₃NCH₂⁺); FT-IR (cm⁻¹): 1599, 1463 (νbpy, m), 1208.7, 1144.7 (νCF₂, vs); m.p.: 125-128° C.

Compound 1b: (NMR data collected in CDCl₃ at 60° C. to increase the solubility): yield (sublimed) 65%; ¹H NMR (500 MHz, CDCl₃) δ 8.69 (2H, d, ³J_{HH}=5.1 Hz, H₆), 8.40 (2H, s, H₃), 7.38 (2H, d, ³J_{HH}=4.2 Hz, H₅), 4.80 (4H, s, bpy-CH₂), 4.06 (4H, t, ³J_{HF}=13.3 Hz, CF₂CH₂); ¹⁹F NMR (470.5 MHz, CDCl₃) δ −80.7 (3F), −119.3 (2F), −121.7 (6F), −121.8 (4F), −122.6 (2F), −123.1 (2F), −126.0 (2F); ¹³C NMR (113 MHz, CDCl₃) δ 73.1 (bpy-CH₂), 68.1 (CH₂CF₂), 119.8, 122.2, 144.7, 149.4, 154.1 (bpy), 105.5-116.2 (C₁₀F₂₁); GC/MS (m/z; EI): 732 (M⁺-OCHC₁₀F₂₁), 198 (C₅H₃NCH₂C₅H₃NCH₂O⁺), 183 (C₅H₃NCH₂C₅H₃NCH₃⁺), 91 (C₅H₃NCH₂⁺); FT-IR (cm⁻¹): 1602.4, 1561.7 (νbpy, m), 1215.0, 1150.5 (νCF₂, vs); m.p.: 140-142° C.

Compound 1c: (NMR data collected in toluene at 90° C. to increase the solubility): yield (sublimed) 63.2%; ¹H NMR (500 MHz, D-toluene) δ 8.51 (2H, d, ³J_{HH}=5.1 Hz, H₆), 8.52 (2H, s, H₃), 6.93 (2H, d, ³J_{HH}=4.2 Hz, H₅), 4.19 (4H, s, bpy-CH₂), 3.59 (4H, t, ³J_{HF}=13.3 Hz, CF₂CH₂); ¹⁹F NMR (470.5 MHz, D-toluene) δ −81.1 (3F), −119.3 (2F), −121.7 (12F), −122.6 (2F), −123.1 (2F), −125.8 (2F); ¹³C NMR (113 MHz, D-toluene) δ 73.5 (bpy-CH₂), 68.2 (CH₂CF₂), 119.6, 121.8, 146.9, 149.9, 157.2 (bpy), 105.0˜116.0 (C₁₀F₂₃); GC/MS (m/z; EI): 732 (M⁺-OCHC₁₁F₂₃), 198 (C₅H₃NCH₂C₅H₃NCH₂O⁺), 183 (C₅H₃NCH₂C₅H₃NCH₃⁺), 91 (C₅H₃NCH₂⁺); FT-IR (cm⁻¹): 1599.4, 1463.7 (νbpy, m), 1208.0, 1150.5 (νCF₂, vs); m.p.: 147-150° C.

### Example 2

### Preparation of Metal Complex (2)

CuBr (0.1 mmol, 14.3 mg) and compound 1a (0.2 mmol, 236 mg) (as a ligand) were charged into a 50-mL Schlenk flask under the N₂ atmosphere. Then FC-77 (a distilled mixture of perfluoroinated solvent whose boiling point range is close to n-C₈F₁₈ and is commercially available from 3M Company, U.S.A.) (4 mL), HFE-7100 (perfluorobutyl methyl ether; C₄F₉OCH₃) (2 mL) and acetonitrile (3 mL) were added into the flask and the mixture was stirred for 16 h to form dark color materials. After evacuating the solvents, the solid Cu complex (2a), [CuBr(ligand 1a)₂], was formed.

### Example 3

### Atom Transfer Radical Polymerization (ATRP) of MMA Under Thermomorphic Mode

The metal complex (2a) (0.1 mmol, 486.3 mg) as it is prepared in the above Example 2, methyl methacrylate (MMA) (10 mmol, 1 g), and 5.5 mL toluene were dissolved in a flask. After the 3 freeze-and-thaw cycles, the reaction temperature was set to 80° C. In the period of 5 min., an initiator ethyl 2-bromoisobutyrate (0.1 mmol) in small amount of toluene, was slowly added into the reaction solution by using the degassed syringe. At the set time intervals of 3 hrs, 6 hrs, 9 hrs, or 24 hrs, the aliquots were taken by the degassed syringe. And the samples were analyzed by ¹H NMR to calculate the conversion. At the end of reaction, the mixtures became the green solution. Then the mixtures were frozen at −10° C. and it was centrifuged for a half hour. The used solid Cu complex (2a) was separated from the solution by decantation. The polymethyl methacrylate (PMMA) was obtained by evacuating the solvent or was precipitated out by adding the excess methanol to the solution. The MW of resulting PMMA was determined by GPC. And the residual Cu content was analyzed by ICP-MS.

### Example 4

### Reuse of the Metal Complex (2a) in ATRP of MMA

Compounds in the molar ratios of [monomer (MMA)][metal complex (2a)][Azobisisobutyronitrile (AIBN)]=200:1:0.5 were used. Toluene and the metal complex which was recovered from the Example 3, were Charged into a 50 mL Schlenk flask under the N₂ atmosphere. The flask was submerged into the 80° C. oil bath. Then the Azobisisobutyronitrile (AIBN) which was pre-dissolved in little amount of toluene was added and reaction was started. After the polymerization, the products were analyzed by ¹H NMR. The yield was 81%. When the fresh CuBr₂ was used to make the Cu complex (2), the polymer thus obtained was similar to that made by the recovered Cu catalyst.

Gel permeation chromatography (GPC) was used to determine polymer molecular weights and molecular weight distributions (PDI) using polystyrene standards (Polysciences Corp.) to generate a universal calibration curve for poly(methyl methacrylate) (PMMA). The measurements were operated on a Waters SEC equipped with a Waters 2414 refractive index detector and two 300 mm Solvent-Saving GPC columns (molecular weight ranges: 1×10²-5×10³, 5×10³-5×10⁵) at a flow rate of 0.30 mL/min using tetrahydrofuran (THF) as solvent at 30° C. Data were recorded and processed using Waters software package. ¹H NMR spectra were recorded on a Bruker Avance DRX-400 spectrometer using CDCl₃ as solvent. Chemical shifts were reported downfield from 0.00 ppm using tetramethylsilane (TMS) as internal reference.

What is claimed is:

1. A fluoro-ponytailed bipyridine derivatives represented by the general formula (1): 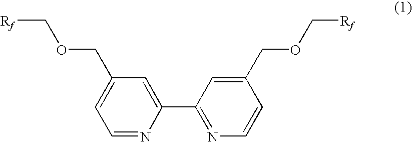
wherein:
each R_{f} is the same or different and represents a fluoro-alkyl group having from 3 to 11 carbon atoms.

2. The fluoro-ponytailed bipyridine derivatives according to claim 1, wherein the R_{f} is the same or different and represents a perfluoro-alkyl group having from 9 to 11 carbon atoms.

3. The fluoro-ponytailed bipyridine derivatives according to claim 1, which is used as a ligand of a metal complex.

4. A metal complex represented by the general formula (2): 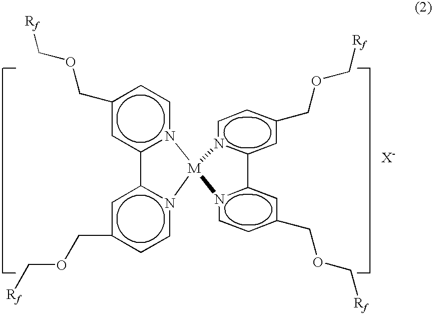
wherein:
each R_{f} is the same or different and represents a fluoro-alkyl group having from 3 to 11 carbon atoms;
X⁻ represents a halogenide; and
M represents a metal selected from the group consisting of Mo, Cr, Re, Ru, Fe, Rh, Ni, Pd, and Cu.

5. The metal complex according to claim 4, wherein the Rf is the same or different and represents a perfluoro-alkyl group having from 9 to 11 carbon atoms.

6. The metal complex according to claim 4, wherein M represents Cu.

7. The metal complex according to claim 4, which is used as a catalyst in an atom transfer radical polymerization (ATRP) under the thermomorphic mode.

8. A method for polymerizing vinyl-containing monomers, which comprises the steps of:
(a) polymerizing one or more of vinyl-containing monomers by using the metal complex according to claim 4 as catalyst at elevated temperature, and
(b) separating the metal complex, formula (2), from the reaction mixture by cooling the temperature of the mixture down to room temperature.

9. The method according to claim 8, wherein the polymerization of one or more of vinyl-containing monomers is an atom transfer radical polymerization (ATRP) under the thermomorphic mode.

10. The method according to claim 8, wherein the polymerization is carried out in the presence of initiator.

11. The method according to claim 10, wherein the initiator is one or more compounds selected from the group consisting of ethyl 2-bromoisobutyrate, (1-bromoethyl)benzene, 1-bromoacetonitrile, 2-bromopropionitrile, and azobisisobutyronitrile (AIBN).

12. The method according to claim 8, wherein the vinyl-containing monomer is selected from the group consisting of alkyl acrylate, alkyl methacrylate, styrenes, and derivatives thereof.

13. The method according to claim 8, wherein polymerization is carried out at a temperature of from 40˜120° C.
